(19) United States
(12) Patent Application Publication
(10) Pub. No.: US 2010/0121315 A1
(43) Pub. Date: May 13, 2010

(54) PERSONAL ACCESSORY FOR USE WITH A PILL

(75) Inventors: Karen I. Trovato, Putnam Valley, NY (US); Pim T. Tuyls, Mol (BE); Anne E. Barschall, Tarrytown, NY (US)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., EINDHOVEN (NL)

(21) Appl. No.: 12/593,634

(22) PCT Filed: Mar. 28, 2008
(86) PCT No.: PCT/IB08/51172

§ 371 Date: Sep. 29, 2009

Related U.S. Application Data
(60) Provisional application No. 60/909,139, filed on Mar. 30, 2007.

(51) Int. Cl. A61K 9/22; A61B 5/07

(52) U.S. Cl. 604/890.1; 600/302

(57) ABSTRACT

A wearable accessory controls (103) and provides security for a pill delivering medical substances and services. An initialization procedure allows decryption information (814) to be loaded into the accessory for each pill and/or decryption information (818) about the accessory to be loaded into the pill. The pill preferably sends encrypted messages to the accessory and the accessory preferably sends encrypted messages to the pill. The pill operates only when the proximity of the accessory is verified or authenticated during the operation of the pill, preferably at frequent intervals. Where the pill is no longer useful, its information can be deleted (1205) from memory in the accessory or other controlling device.

### FIELD OF THE INVENTION

The invention relates generally to the field of medical diagnosis and treatment, and more specifically to control of pills.

### BACKGROUND OF THE INVENTION

The following related applications are incorporated by reference:

U.S. Provisional Application 60/644,538 filed Jan. 18, 2006 (ID778933—Docket US050030 and PCT/IB06/050160 filed Jan. 16, 2006)

U.S. Provisional Application 60/644,539 filed Jan. 18, 2006 (ID778932—Docket US050028 and PCT/IB06/050157 filed Jan. 16, 2006)

U.S. Provisional Application 60/644,540 filed Jan. 18, 2006 (ID778931—Docket US050027 and PCT/IB06/050156 filed Jan. 16, 2006)

U.S. Provisional Application 60/644,518 filed Jan. 18, 2006 (ID779006—Docket US050029 and PCT/IB06/050159 filed Jan. 16, 2006)

U.S. Provisional Application 60/606,276 filed Sep. 1, 2005 (US040322—and PCT/IB05/052820 filed Aug. 29, 2005)

U.S. Provisional Application 60/605,364 filed Aug. 27, 2004 (US040321—and PCT/IB05/052771 filed Aug. 24, 2005)

“Unique and Tamperproof ID for Electronic Pill with Secure Communication for Reporting and Control” a patent application by Trovato et al. filed concurrently herewith, U.S. Provisional Application 60/909,146 (ID778792).

The related applications share at least one inventor with the present application. They are not admitted to be prior art. Nor are any other admissions made with respect to the related applications.

The following additional patent documents are also incorporated by reference:

U.S. patent application Ser. No. 10/497,257 filed Nov. 28, 2002 published as US 20050051351 (Docket PHNL010859)

U.S. patent application Ser. No. 10/497,264 filed Nov. 28, 2002 published as US 20050021993 (Docket PHNL010858)

### DEFINITIONS

As used herein, the term

“pill” shall include any sort of ingestible delivery unit. As discussed in the prior applications a “pill” might deliver a variety of substances or services;

“substance or service” shall include medications, non-medicinal substances, contrast agents, liquids, chemicals, radiological agents, imaging markers, robotic operators, screening, diagnosis, therapy, sensing devices, storing and reporting data such as compliance data, and/or other interventions, including possibly multiple examples of the foregoing. While examples are innumerable a few might include delivery of hormones, pumping insulin, or defibrillation;

“ingestible” will normally mean swallowed, but may also include being inserted into the body by some other means;

“clinical setting” shall include any supervised treatment facility such as a hospital, doctor's office, senior center, senior assisted and independent resident living, or nursing home.

A disadvantage of known pills is that they lack security necessary to preserve medical confidentiality. Another disadvantage of known pills is that an outside controller cannot send respective, individual commands to such known pills. Still another disadvantage is that there is no validation that the substance or service is locked to a particular patient, thus assuring that the correct substance or service is delivered to the correct patient.

### SUMMARY OF THE INVENTION

It is desirable to provide personalized security for pills, both for the purpose of preserving medical confidentiality and for the purpose of improving control of which patient gets a particular medical service or substance.

Encryption technology is provided for a pill. A wearable personal accessory is coupled with the pill for engaging in encrypted communication with the pill. The pill does not release medical substances or perform medical services unless the wearable accessory is present. If the medical substance is a controlled substance, only the correct patient will receive the substance.

### BRIEF DESCRIPTION OF THE DRAWINGS

Various objects, advantages, and embodiments will be apparent from the following.

The invention will now be described by way of non-limiting example with reference to the following figures:

FIG. 1A shows a patient with an ingested pill and a first wearable accessory

FIG. 1B shows a patient with an ingested pill and a second wearable accessory FIG. 2 is a schematic of a pill

FIG. 3 is a schematic of an accessory

FIG. 4 shows a hospital bracelet acting as a wearable accessory

FIG. 5 is a schematic of a PUF unit for use in security.

FIG. 6 is a flow chart showing a security initialization procedure.

FIG. 7 is a flow chart for a pill waking up and recognizing an accessory.

FIG. 8A shows a trusted enrollment system.

FIG. 8B shows an example of a system for matching enrolled devices with one another where each has a PUF providing an, ea ID and Secret.

FIG. 9 shows an example of operation of a system in accordance with the invention in tabular form.

FIG. 10 is a table illustrating operation in the situation of a pill that is missing or malfunctioning.

FIG. 11 is a table illustrating a situation of an accessory that is missing or malfunctioning.

FIG. 12 is a flow chart relating to managing memory in conjunction with the invention.

FIG. 13 shows a packaged system with an accessory and associated pills.

FIG. 14 is a schematic illustration of an accessory communicating with a plurality of pills, each having a unique ID and secret.

### DETAILED DESCRIPTION OF EMBODIMENTS

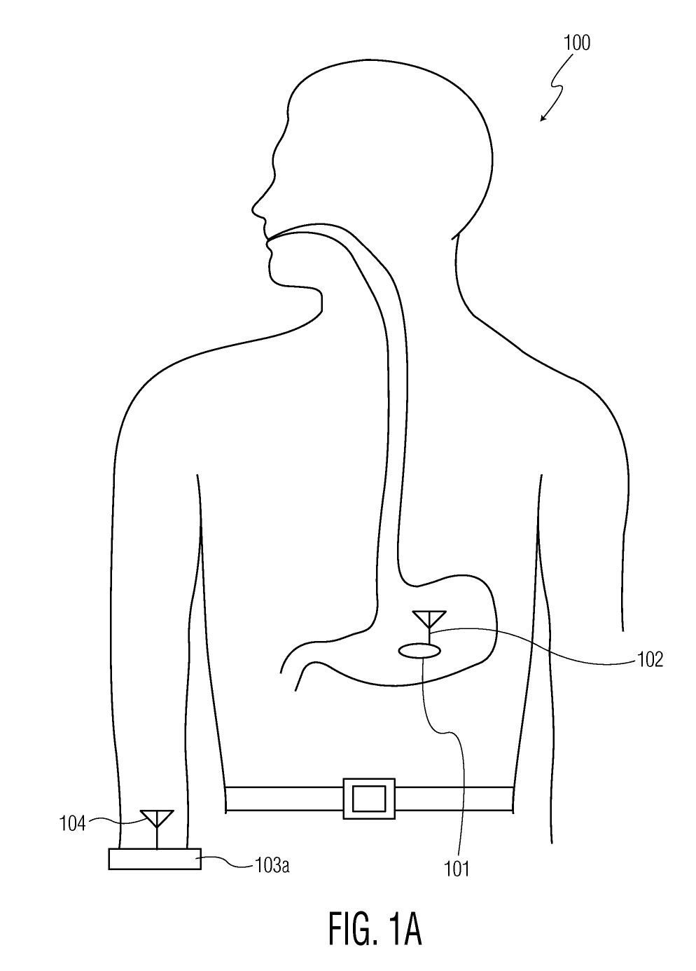

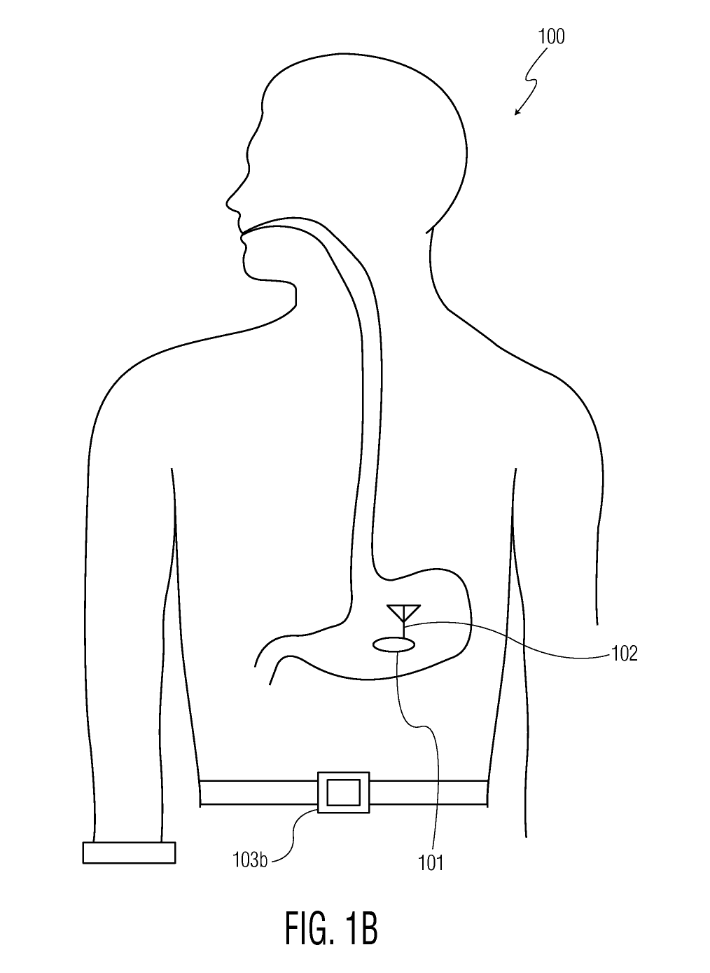

FIGS. 1A-1B show a pill 101 ingested by a patient 100. The pill 101 has an internal antenna 102 for communicating with the exterior. The antenna 102 is schematically illustrated as a wireless communication. The pill 101 is shown in the patient's stomach, but it may be anywhere in the alimentary canal, e.g. in the small or large intestines. While only one pill is shown, it is understood that multiple pills may be active in the patient at one time, as needed. Pills with a longer term or specialized substances or services may be injected or surgically implanted. In FIG. 1A, the patient 100 is wearing a bracelet 103a which is one example of an accessory for communicating with the pill 101 via antenna 104.

Although the accessory in FIG. 1A is a bracelet 103a, it is well known in the accessory art, there are many other types of accessories, such as necklaces, headbands, belts, broaches, name tags, rings, neckties, earrings, and so forth. The accessory is preferably located near the pill to reduce required transmission power. For example, to reach a pill in the alimentary tract, it may be preferred to use an accessory that pins to clothing or is attachable at the waist, for instance by being hooked on a belt or belt-buckle 103b, as shown in FIG. 1B. The belly button area is a particularly advantageous area for the accessory, because that area will be nearest the pill while the pill is in the stomach. In extreme cases of concern, for instance where controlled substances are involved, the accessory may be implanted in the patient. A patient may be wearing or otherwise carrying more than one accessory at a time, where the patient is taking more than one medication. The patient's garments might even be wired for communicating with the pill. It is also possible for the security functions described herein to be effected using a removable module that is attachable to an accessory functioning as a carrier for the module. As used herein, the term “wearable accessory” or just “accessory” includes all of these possible carrier modalities, including a module attachable to another accessory.

FIG. 2 shows a diagram of an exemplary pill 101 for delivering a medical substance. The pill has an antenna 102. While this particular pill 101 is configured to deliver a medical substance, this is just one possibility. A similar pill might be used to monitor and store drug usage or dosage data and report a patient's compliance with a particular prescription or dosage. A similar pill might deliver medical services not related to releasing a substance, such as operating a sensing device or manipulating a tool.

The pill 101 has a start timer mechanism 202 for triggering a timing circuitry 203. The start timer mechanism 202 is for example, the external coating 201 of the pill dissolving in digestive fluids and initiating an electrolytic current or a signal received via the antenna 102. If the former is the starter timer mechanism 202, the pill is optionally configured to be completely turned off until its external coating dissolves. Alternatively the pill may engage in occasional polling of its environment looking for triggering signals or it may have passive reception ability, where it receives enough power from an incoming signal, low bandwidth signal to wake up the higher power, higher bandwidth receiver. This dual power level is used in communications items such as PicoRadios and Zigbee, which are described in the following references.

### REFERENCES

J. M. Rabey et al. “12.3 PicoRadios for Wireless Sensor Networks—the Next Challenge in Ultra Lower Power Design”, IEEE International Solid States Circuits Conference (ISSCC February 2002) published at http://bwrc.eecs.berkeley.edu/Publications/2002/presentations/isscc2002/12_{—}3_text.pdf
G. Legg, “ZigBee: Wireless Technology for Low-Power Sensor Networks” (May 6, 2004) http://www.techonline.com/community/related_content/36561
The official website for ZigBee is at http://www.zigbee.org/en/index.asp

The timing circuitry 203 cooperates with the release controller 204 to govern release of the medical substance, via valve or release hatch 205. The controller 204 has a security mechanism within such as for example, PUF technology. PUF technology for creating tamper resistant bit strings is disclosed in international patent application WO 2004/105125 A2, published Dec. 2, 2004, and entitled “Semiconductor Device, Method of Authenticating and System” which designates the U.S. and is incorporated herein by reference. Security technology preferably allows the pill 101 to communicate with the outside world using encrypted messages. The controller 204 controls release mechanism 206 to release the medical substance from the reservoir 207. Release is possible in accordance with many criteria, such as timing, an internal release profile, and/or commands from the outside. As will be discussed further below, the pill 101 will not release any medical substance or perform any medical service until it verifies that an appropriate accessory or other security is present. The pill 101 is programmed with a set of commands that it is able to carry out. The pill is optionally programmed to be able to receive these commands from more than one device, such as a work station or scanner in a clinical setting as well as the wearable accessory.

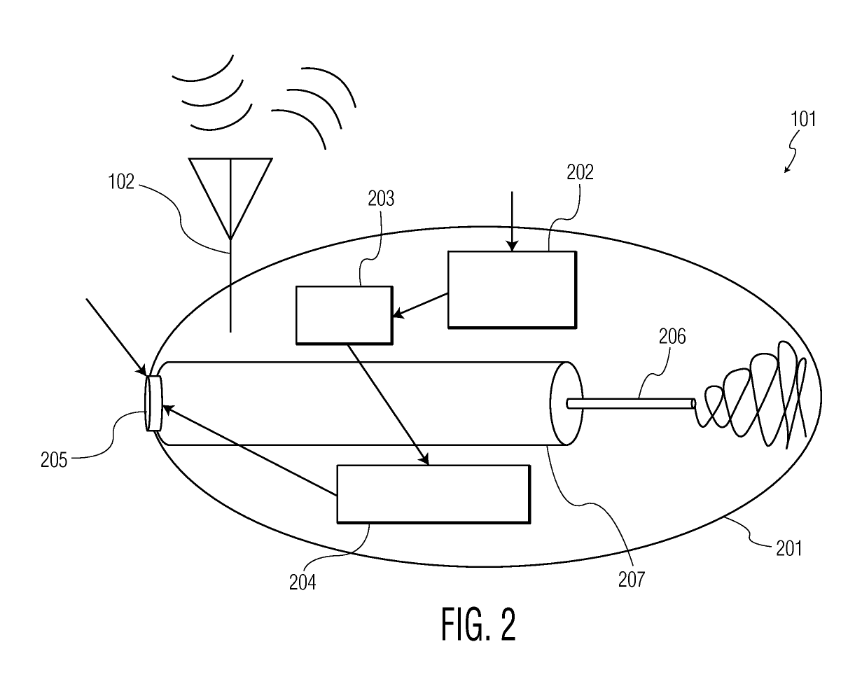

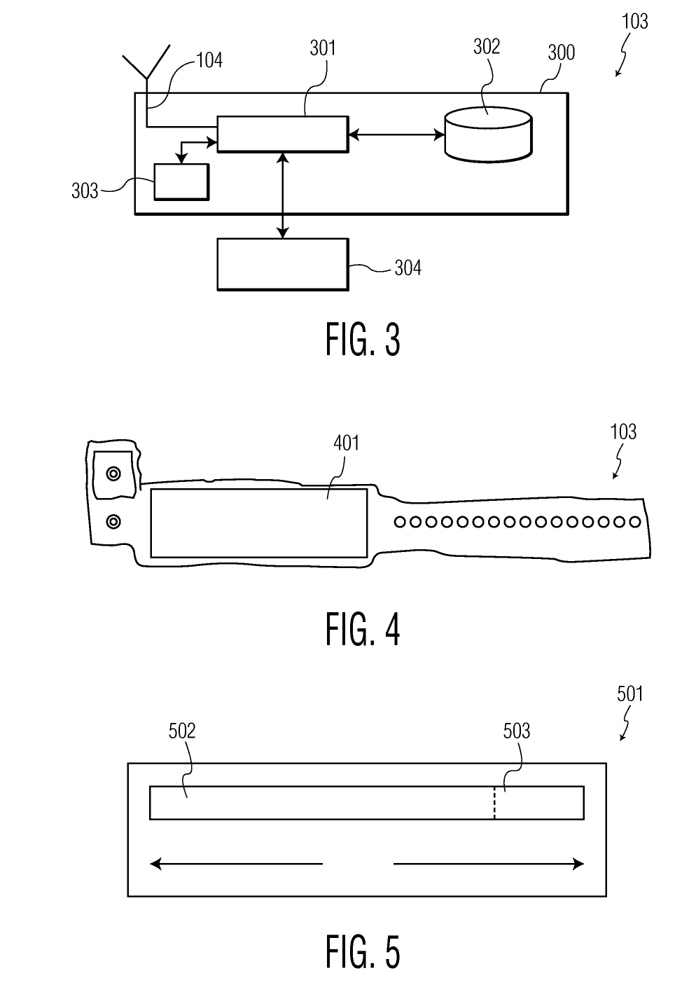

FIG. 3 shows an accessory 103 (such as for example bracelet 103a in FIG. 1A or belt buckle 103b in FIG. 1B) including a module 300 for securing communication with the pill 101. The module 300 is particular to one patient. Preferably, one of the communication related functions undertaken using the module 300 assures that the pill operates only for that particular patient. This is accomplished for example using an authentication operation, as known in the digital security arts. The module 300 is contained in a housing for integration or attachment with accessory 103 or integrated into a carrier, e.g. belt, bracelet, or necklace without a housing. The housing, if any, may be decorative, for instance having the appearance of a piece of jewelry or contributing to the overall appearance of the accessory 103 as jewelry. The module 300 includes a processor 301 in communication with a memory 302. The memory 302 for example contains data and/or executable code for use with the processor 301. Optionally, the module 300 has its own secure identification module at 303, which implements PUF technology for example, as discussed below, or some other form of security. The secure identification module is optionally effected or integrated within the processor 301 and memory 302. The module 300 also optionally contains an external control 304. This optional external control 304 may be as simple as a power on/off, or may include a knob for regulating dosage of medical substances—or it may be sufficiently sophisticated as to have a small touch screen, a display with control buttons or even a keyboard—all depending upon how much functionality is desired.

More information about how a control device communicates with a pill or pills can be found in prior applications.

FIG. 4 shows an exemplary embodiment of an accessory 103. In this case the circuitry of FIG. 3 is hidden within the nameplate 401 of a traditional hospital bracelet. The bracelet may be provided in a package with a set of pills, wherein the bracelet and the pills are pre-programmed prior to insertion into the package to recognize each other, and to engage in encrypted communication with each other. Alternatively, the pills may be distributed separately and the bracelet is programmable to recognize pills as they are prescribed. This latter embodiment would be preferable for a patient taking more than one type of pill, to avoid the patient having to wear more than one accessory. Some patients, taking many medications would require too many accessories if each pill were required to have a separate accessory. In addition, a single accessory can coordinate delivery of substances or services. For instance, some medications may be incompatible so that one is not to be delivered until the other is completely dispensed and absorbed by the body. Or several pills delivering the same substance might need to be coordinated to assure continuity of dosages, without overlap. Also, for instance, if it were desired to image with and without a contrast agent, a pill releasing a contrast agent might wait until a pill with imaging equipment had taken a first set of images before releasing the contrast agent. Information necessary for such coordination might come through the personal accessory 103, which would allow for simplification of the pills.

In general, it is desirable for portable medical devices, such as a pill 101 or an accessory 103, to be as simple as possible. Within this constraint, many designs are possible based on the particular functions desired by the pill 101. Typically, since the pill 101 is preferably small for facilitating swallowing and cannot be readily modified once ingested, it is advantageous to put more control functions in the larger accessory 103, which can also be replaced if damaged. Nevertheless, there may be instances in which more sophistication is desired within the pill 101.

Secured communication between the pill 101 and the accessory 103 might take many forms. Preferably, encrypted messages are sent within a system that includes an accessory 103 and one or more pills 101. One type of encryption uses PUF technology. PUF technology includes an N bit storage unit 501 as shown in FIG. 5. The unit 501 includes a storage area that has a publicly accessible ID 502 and a secret number 503. The secret number is used to encrypt messages. One device (“the querying device”) can query another device (“the receiving device”) with PUF technology. In response to the query, the receiving device reveals its non-secret ID 502. If the querying device already stores or has permission to access (such as from a remote server) the secret 503 corresponding to that non-secret ID 502 of the receiving device, then the querying device can encrypt messages using that secret 503. The receiving device then can decrypt, and thereby recognize, commands from the querying device, using the receiving device's secret and therefore trust the querying device.

One possible scenario is that the pill 101 sends only encrypted messages, but can recognize unencrypted messages. In this scenario, the wearable accessory 103 is programmed with secret information that allows it to decrypt the pill's encrypted messages. In the case where the pill 101 sends an unencrypted message, the wearable accessory 103 optionally includes some other type of information that permits the pill 101 to authenticate it. In the digital security arts, there are many examples of authentication between two devices.

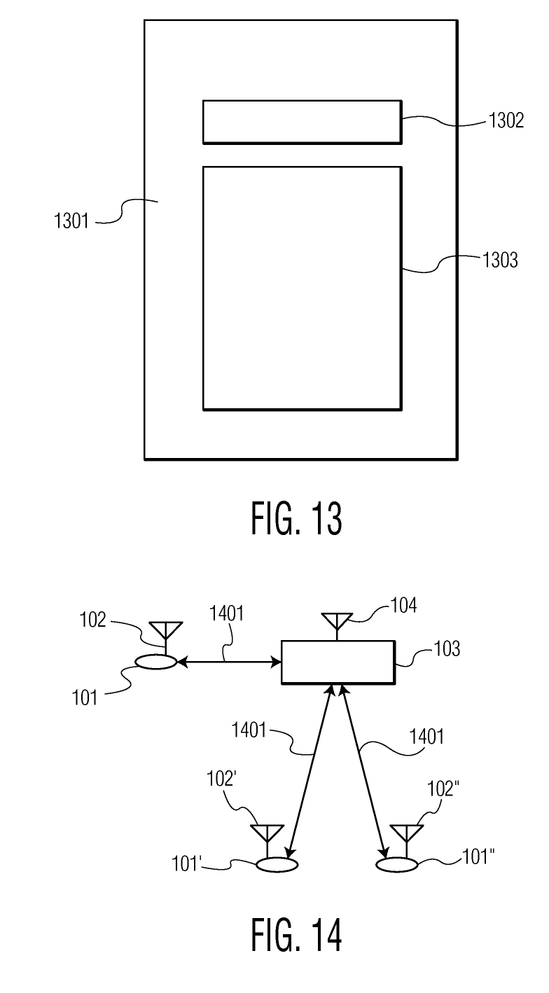

A wearable accessory and a bottle of pills may be sold as a set. This is shown at FIG. 13, where a package 1301 contains an accessory 1302 and a container 1303 of coordinated pills. The package 1301 is a box or other packaging. The pills and/or accessory are optionally in a blister pack. In such a set, the accessory and pill are pre-programmed to recognize one another. In this case, all the pills share the same encryption key—or they each have a separate key that is pre-stored in the accessory 1302. Assigning a separate key to each pill, such as with the PUF technology, improves security. Assigning a separate ID to each pill ensures that each pill can be addressed and controlled individually. More than one pill might be in the alimentary canal at a time, either because multiple pills are required to carry out desired functions or because it may take 24 hours or more for a pill to work its way through the body. Where multiple pills are expected in the body, assigning some sort of individual identification to each pill allows the accessory to coordinate release between each pill to maintain doses or other services such as reporting compliance for one or more delivered substances. FIG. 14 shows in a schematic form, an accessory 103 communicating with a plurality of pills 101, 101′, and 101″, via antennas 102, 102′, and 102″, with reference numerals of like devices being the same as in FIG. 1. The pills 101, 101′, and 101″ are understood to be within a human body (not shown in FIG. 14). Communication between accessory 103 and pills 101, 101′, and 101″ occurs via messages 1401, where each message 1401 is encrypted using the key of the respective pill 101, 101′, and 101″.

The accessory may also give out a warning message if it notes that too many pills have been ingested at one time—or if the patient has forgotten to take one—as well as monitoring, reporting data such as compliance, controlling, and coordinating substances or services delivered by two or more pills. Those substances or services might be the same or different. A bottle may be sold with a set of coordinating pills designed to deliver a variety of substances and services customized to a particular patient, together with the pre-programmed wearable control accessory. Pills may be controlled to prevent incompatible medical substances or services from being released at the same time, or to maximize the effect of substances or services that are supposed to be released at the same time. More information about such coordination can be found in prior applications, with respect to other types of control systems.

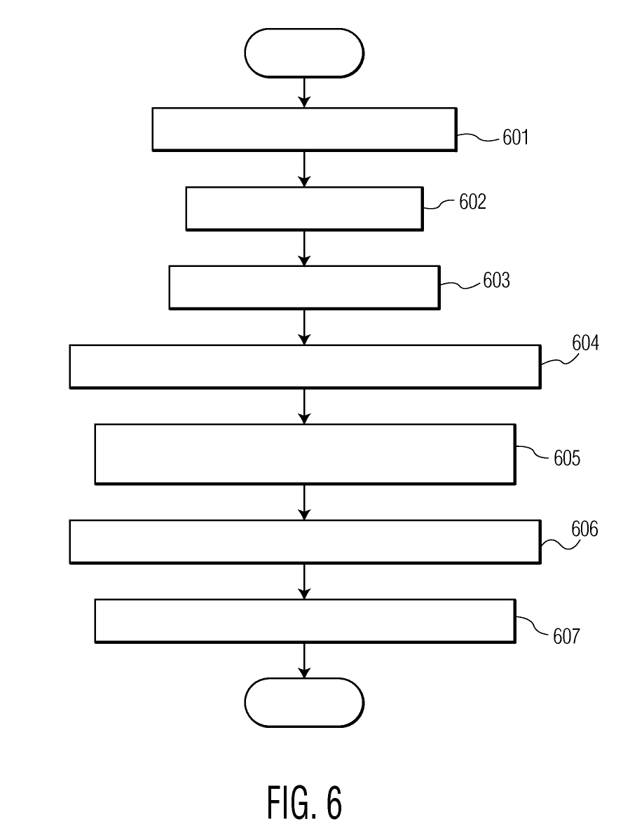

In another scenario, a patient purchases a permanent or periodic accessory, which is re-programmed every time a new pill or group of pills is added to the patient's treatment profile. Accordingly, a pharmacy for example, reprograms the accessory for each new pill. FIG. 6 shows a procedure for this scenario. The accessory is received by the patient at 601. Then the prescription is retrieved at 602. This prescription is delivered in one of many ways, including: a traditional paper prescription; a secured program within the accessory; or separately to the pharmacist, by phone, fax or via secured electronic communication. Then one or more pills are retrieved from storage at 603. The pills are queried by the pharmacist or other matcher's electronic system to retrieve each publicly available IDs at 604. The pharmacist, or programming device, then obtains the secret information associated with the pill at 605. This secret information is available via a local, regional, or central database, with which the pharmacist or programming device has secure and authenticated communication. Alternatively, the secret information is delivered on some medium—such as a bar code—to the pharmacist along with the pills or communicated to the pharmacist or programming device at the time the pills are ordered from a manufacturer or wholesaler. Subsequently, the secret information for each pill is loaded into the accessory at 606 and the accessory is returned to the patient, along with one or more pills at 607.

FIG. 6 assumes that the wearable device or accessory is programmed with secret information about the pill. Optionally, it is desirable to program the pill with identifying or secret information about the accessory. A decision of which device ought to be programmable to recognize the other might depend on considerations of desired price and size of each, or upon which device might be considered more susceptible to tampering.

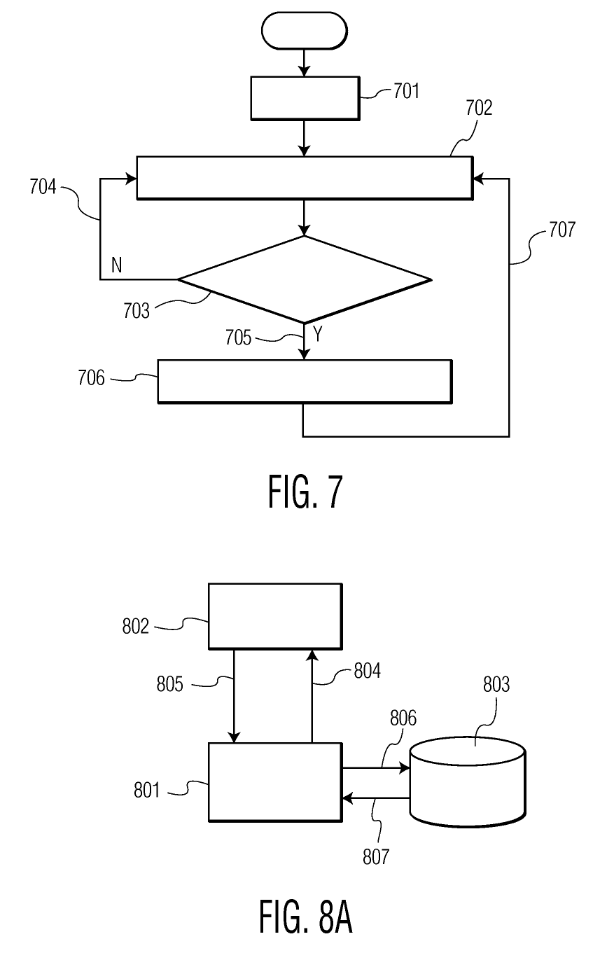

FIG. 7 shows a flowchart of an exemplary process by which the pill recognizes the accessory. Optionally, at 701, the pill wakes up. As discussed above in regard to FIG. 2, it is possible for the pill 101 to continuously test the environment for the correct conditions to begin acting. At 702, the pill must have verification that the accessory is nearby. This can be assured by having the pill poll for the accessory's existence, or by the pill verifying regular communication from the accessory. In the former case, for example, the pill transmits a message such as ‘are you there?’ to the accessory—encrypted with the pill's secret. The accessory then responds affirmatively, e.g. ‘A responding’ or ‘ACK-A’ in a message encrypted with the pill's secret. This occurs for example every N seconds, so that the pill is assured that it is continuing to act on the proper patient. An optionally more secure transmitresponse set of messages includes a slightly altered transmitted message, so that the response from the accessory cannot be duplicated by an imposter. For example, the pill transmits ‘are you there A?’ and expects to receive a response: ‘yesA’ or ‘ACK-A’. In the latter case, for instance, the pill simply receives, expecting a message encrypted with its secret. Such a message says for example: ‘accessory is alive,’ or provides some useful, dynamic information such as ‘time is now 10:31.’ The dynamic information would be more difficult to impersonate. The security process in the pill alternatively is powered passively by the power of the message signal from the accessory, such as in an RF_ID tag. After the presence of the accessory is detected, at 703, an authentication process occurs. If authentication is not successful, at 704, the pill keeps looking for an accessory. Authentication may be in accordance with a number of known algorithms in the digital security arts. If authentication is successful at 705, a substance or service is delivered at 706. Per 707, the pill continues looking for the accessory at given time intervals, even after the first authentication, to make sure that the delivery of substances or services is still appropriate. To halt delivery of the substance or service, a medical service provider removes the accessory from the patient. The accessory is optionally programmed with a HALT command which can be sent to the pill.

Commands encrypted with the pill's secret and sent to the pill from the authorized accessory include for example:

Setting a substance release pattern;

Halting delivery;

Causing a burst of substance;

Causing a particular service action;

Requesting reports from the pill; and

Supplying current date information to the pill

When the pill recognizes an encrypted command, it can trust the accessory. Using encryption in accordance with the pill's own key as authentication has the advantage that any device having that key can access the pill. So, for instance, the pill may be controlled by either the accessory or a remote workstation or both.

While FIG. 7 is drawn with respect to only one pill, it is understood that the same process may occur in parallel in several pills at once.

In addition, the pill is optionally capable of providing authentication to the accessory. For instance the pill is optionally programmed, preferably in write-once memory, with a secret of the accessory, ensuring that communication from the pill is only understood when decrypted by the accessory. The accessory can decrypt all incoming messages from the pill because the incoming messages are encrypted by the pill with the accessory secret. Although the message will be wirelessly broadcast, it will decrypt to a recognizable command only by that specific accessory. The individual pill would have to include its key within its ‘return address’ within the message so that the accessory can calculate the encryption of subsequent messages for this specific pill. Alternatively, the accessory might be pre-programmed, also in write-once memory, with the pill's ID and key so that only the ID is used as the ‘return address’, which is encrypted and then communicated wirelessly.

Although potentially less secure, since a “back door” results, the accessory may be programmed to allow secured override by a workstation in a clinical setting, to permit a treating medical service provider to alter treatment orders in real time.

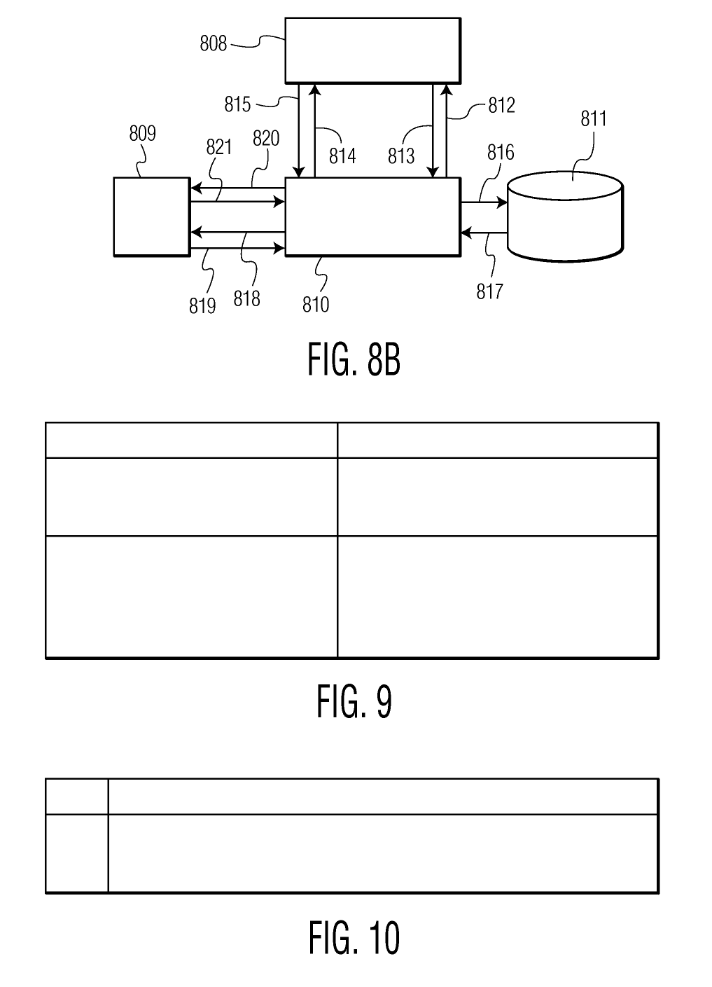

FIGS. 8A and 8B show the components and processes of Enrollment and Matching Systems, which are described in more detail below.

FIG. 8A shows the Trusted Enrollment System 801. This system is typically used by a manufacturer of devices to be enrolled and comprises a computer, memory and communication means (not shown) for communicating with an Enrolling Device 802 and a Master Database 803. An Enrolling Device 802 is an accessory, a pill, or any other device that might be used to communicate securely with these devices.

The enrollment process begins in FIG. 8A when the Trusted Enrollment System 801 sends a message 804 such as ‘SEND ID and Secret’ to the Enrolling Device 802. The Enrolling Device then sends message 805 which includes the ID and Secret which are enclosed in the Enrolling Device 802. The Trusted Enrollment System 801 then transfers the information via message 806 to the Master Database 803. The Master Database 803 may be as simple as stored data or as complex as a remote database management system with server. Further, the Master Database optionally includes other information such as the Medication type, Manufacturer, Expiration Date, Lot number, Barcode or other information. This information can be used in an emergency so that emergency room doctors or ambulance personnel can immediately determine the type of medications taken by a patient.

Preferably, the Enrolling Device 802 is programmed to provide the Secret only one time. This ensures that once the Enrolling Device 802 is enrolled, the Secret cannot be released again. Another alternative may be that a second request for the Secret will cause the Enrolling Device 802 to shut down permanently, such as if a security breach is underway. Communication with the Enrolling Device 802 may be unencrypted if performed in an environment free from eavesdroppers, but may also use a pre-programmed encryption scheme, or one that is a function of lot number if this is stored in the Enrolling Device 802. The Master Database 803 verifies that the ID, optionally including other attributes stored in the Enrolling Device 802 such as lot number, product bar code, manufacturer, medication type, etc. is unique, or otherwise the Enrolling Device 802 should be rejected. After the ID and Secret are sent from the Enrolling Device 802 to the Master Database 803 via message 806, the Master Database 803 returns a message 807 indicating ‘OK’ or ‘Reject’.

FIG. 8B shows an example system for matching Enrolled Devices with one another where both have a PUF providing their ID and Secret. This system may typically be used by a manufacturer of enrolled devices or by an authorized pharmacy. In this scenario, the enrolled devices are an Authenticator 808 stored within an accessory such as a bracelet and a Pill 809. A Trusted Matching System 810 communicates with the Authenticator 808, Pill 809 and Master Database 811. The Trusted Matching System 810 sends a message 812 to the Authenticator 808 requesting its ID. The message for example may be ‘SEND ID’, and may be unencrypted. The Authenticator 808 will return the stored ID via unencrypted message 813, which might look like: 11235813. Similarly, the Trusted Matching System sends a message 820 to the Pill 809 requesting the pill's ID. The Pill then returns the ID via message 821, which might look like: 224610162. Standards exist to denote start and stop components of the message so that the ID numbers do not have to be the same length. It is also clear that a single unencrypted wirelessly transmitted ‘SEND ID’ message might return the IDs of both devices if they are both within communication range of the Trusted Matching System 810. Collision detection protocols, checksums and acknowledging messages can ensure clear transmission of the ID numbers.

Once the set of IDs are acquired, and possibly checked against the expected number of entries, the Trusted Matching System 810 then sends a query message 816 to the Master Database 811 requesting the secrets of the various IDs. Since this link is one of the most vital, it is assumed that any one of the numerous authentication and encryption schemes available ensure secure and valid communication between the computer within the Trusted Matching System 810 and the Master Database 811, particularly if the Master Database is accessed via a network. The Master Database, or server, that manages the database then returns the respective secrets via message 817. The Master Database may further forward information about the type of device that relates to the stated ID, so that particular protocols can be performed, expiration dates can be set, advisories reported, etc. The Trusted Matching System then sends messages to the respective enrolled devices to cause them to store secrets for the required enrolled devices. In this example, the Trusted Matching System 810 sends a message encrypted with the Authenticator's secret to Authenticator 814 with message 814 stating ‘Store Secret 4525136 ’, the Pill's secret. Optionally to assure valid transmission and execution, the Authenticator 814 may send an acknowledgement 815, encrypted with the Authenticator's secret, that the ‘storage 4525136 is completed’. Message 814 might also contain information about substances or services to be delivered by the pill 809. Such information may be necessary for controlling and/or monitoring functions to be performed later by the accessory.

The Trusted Matching System 810 then sends a message encrypted with the Pill's secret to Pill 809 with message 818 stating ‘Store Secret 3542751 ’, the Authenticator's secret. Optionally to assure valid transmission and execution, the Pill 809 may send an acknowledgement 819, encrypted with the Pill's secret, that the ‘storage 3542751 is completed’.

When we describe a ‘Master Database’, it is not necessarily the complete directory of all enrolled devices ever made. It may be a subset that is confined to the devices purchased within a facility such as a nursing home. This has the advantage that enrolled products brought in from the outside cannot be accidentally or intentionally substituted for authorized medications for a particular person. A clearinghouse containing all known enrolled devices might be maintained as a backup.

In this way, each of the pill and the accessory are programmed to send encrypted messages to the other according to the encryption that the other expects. While only one pill is illustrated, it is understood that multiple pills might appear in the system at the same time or sequentially.

FIG. 9 shows an example operation of a system in accordance with the invention in tabular form. In this case, the pill has expired medication. The pill might learn this either because it has its own internal timer, or from comparison of its own expiration date with a date supplied by the accessory. The pill therefore sends an encrypted message, using its own encryption key, indicated by italics, saying that its medication has expired. In response, the accessory sends back an encrypted message, also using the pill's encryption key authorizing the pill to fail, or simply not release medication. The accessory can then give an error message. The error message might be in the form of beeping, color change, or a message on a local display. Alternatively, the accessory might communicate with a nurse's station.

Typically, the accessory can have larger or batteries and larger storage space than the pill. The batteries of the accessory may also be recharged or exchanged. This is more difficult with the pill, which may be inside the patient's body, or sealed with a coating. Therefore the accessory may be better able to relay pill status by communicating with the nurse's station—or by becoming visibly or audibly active—than the pill is.

FIG. 10 shows a situation where the pill is either missing or malfunctioning, and the accessory has not received a report indicating either activation or timely delivery of a substance or service. Again the response may be to issue a local or remote alarm. In response to the alarm, a new pill can be dispensed to the patient. Alternatively, if an unexpected pill is found in the patient, this can also be reported to a nursing station or central database. It may be that the unexpected pill was supposed to be in a different patient, who may need to be located and given a replacement pill.

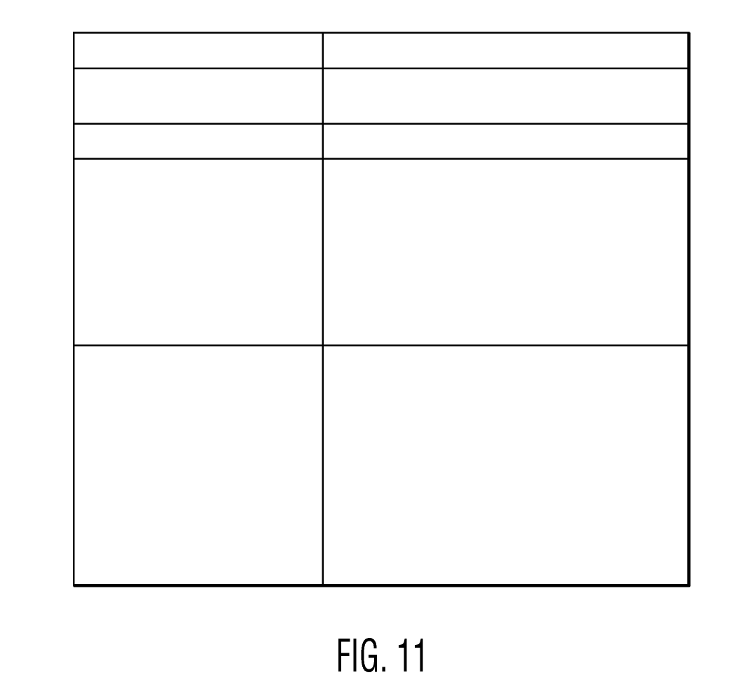

FIG. 11 shows an embodiment of a situation in which the pill sends an encrypted message using its own encryption key requesting activation row 2, col. 1, but gets no response from the accessory, row 3, col. 2. After a pre-set period of time, for instance five minutes, the pill stops its current actions, particularly delivering substances or services, and sends out an unencrypted error message saying that it has not found the required accessory at row 4, col. 2. This message may be received by any authenticator, such as another patient's accessory, or a workstation. The receiving authenticator may raise an alarm either locally, or by forwarding the message elsewhere, with an indication that it has been forwarded at row 5, col. 2. Typically, this will work best in a clinical setting where there are enough accessories around that a pill can send a warning message to other devices that may relay information to an appropriate receiver where the problem can be solved find something to communicate with. The accessories may engage in packet hopping to communicate throughout larger areas. Ultimately, a network of stations might be set up outside the clinical settings so that messages from pills might be received.

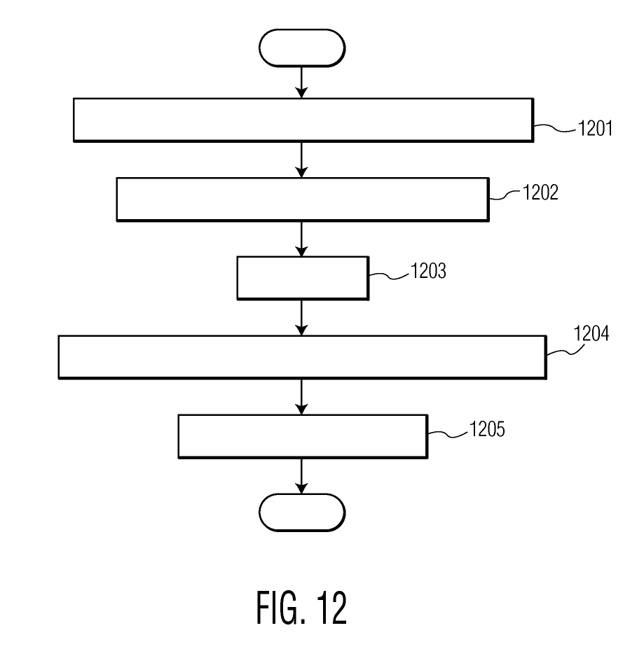

In general, it may be desirable for the accessory to keep a record of which expected pill has been activated, and erase that pill from memory after a given period of time, say 48 hours, when it is reasonably certain that the pill has been eliminated. This will allow for smaller memory units within the accessory and potentially reduce cost while increasing expected lifetime. Other types of controlling devices, not just the accessory, may similarly delete pill records from their memories. FIG. 12 shows a flow chart of this operation. First, at 1201, the accessory receives and stores pill identification for one or more pills. Then at some later time, possibly much later, the accessory receives an indication at 1202 that some pill—from the set for which the accessory is storing identifying information—has been activated. The accessory then sets a timer at 1203. There will have to be a timing mechanism for each pill that has been activated. This can be done with counters and software loops—or such as by setting a pre-defined number of ‘ticks’ in an array. Once the number of ticks has been reached, the elapsed time has been reached. The accessory then determines at 1204 that a pre-set threshold time, such as 24 or 48 hours, has been reached. Some pills will have exited the digestive tract, exhausted their capacity, or finished their operations by this time. A different threshold might be set, depending on medical needs and pill capabilities. The determination that the threshold has been reached allows the accessory to delete the pill from memory at 1205. This deletion may include security information, such as ID and secret key, and/or information regarding the substances and/or services that the pill was expected to deliver.

Once the pill is deleted from memory, the accessory can no longer communicate with the pill. The pill will, therefore, cease to dispense substances and/or services, in accordance with the embodiments discussed above. The deletion from memory thus serves both a security purpose and also a memory economization purpose. Alternatively, the pill's identification, secret key, etc. might be deleted from memory after some other determination, such as that the pill has reached a medicine's potency expiration date or the patient's medical condition has changed so that the pill is no longer needed. Herein, the pill will be stated to be no longer “useful” when some criterion, such as time threshold, expiration, and/or medical prescription changes, makes deletion from memory desirable. Deletion of pill information is especially advantageous when the controlling device is a wearable accessory, since wearable accessories need to be small and cheap. Nevertheless, deletion of pill information that is no longer useful can still be desirable in a larger medical control device, such as a workstation, and for efficient storage of a ‘Master Database’. For example, a Master Database is more compact and more quickly searchable if it only contains those pills that are currently relevant (i.e. non-expired and never used) rather than a copy of all pills created since those that were first manufactured. Deleting expired or used pills will also reduce the risk that a new, randomly generated ID will match an existing pill's ID, thus reducing waste.

From reading the present disclosure, other modifications will be apparent to persons skilled in the art. Such modifications may involve other features which are already known in the design, manufacture and use of medical devices and which may be used instead of or in addition to features already described herein. Although claims have been formulated in this application to particular combinations of features, it should be understood that the scope of the disclosure of the present application also includes any novel feature or novel combination of features disclosed herein either explicitly or implicitly or any generalization thereof, whether or not it mitigates any or all of the same technical problems as does the present invention. The applicants hereby give notice that new claims may be formulated to such features during the prosecution of the present application or any further application derived therefrom.

The word “comprising”, “comprise”, or “comprises” as used herein should not be viewed as excluding additional elements. The singular article “a” or “an” as used herein should not be viewed as excluding a plurality of elements. The word “or” should be construed as an inclusive or, in other words as “and/or”.

1. A pill (101) comprising:
a wireless transceiver (102);
at least one device (205) adapted to deliver a substance and/or service; and
at least one processor (204) adapted to perform operations, the operations comprising:
seeking an accessory for initiating (702) secured communication; and
refraining or blocking (704) delivery of the substance and/or service if the accessory is not found.

2. A medical security device comprising:
at least one wearable accessory (103, 105) the accessory comprising:
at least one wireless transceiver (104);
at least one memory device (302) for storing data and/or executable code; and
at least one processor (301, 303) adapted to execute operations including
sending at least one message to at least one pill (101) via the wireless transceiver; and/or
receiving at least one message from the at least one pill (101) via the wireless transceiver.

3. The device of claim 2, wherein the operations further comprise receiving (606) encryption related information for said at least one pill, wherein the sending at least one message comprises encrypting and the receiving at least one message comprises decrypting with the received encryption related information.

4. The device of claim 2, wherein the operations further comprise storing encryption related information unique to the wearable accessory for said at least one pill, wherein the sending at least one message comprises encrypting and the receiving at least one message comprises decrypting with the stored encryption related information.

5. A system comprising:
at least one ingestible pill resident in the alimentary tract of a human body (100); and
at least one wearable accessory, the accessory comprising at least one wireless transceiver (104), at least one memory device (302) for storing data and/or executable code, and at least one processor (301, 303) adapted to execute operations including establishing secure communication with at least one pill (101) for enabling pill functionality, and monitoring or controlling the pill via encrypted messaging.

6. The system of claim 5, wherein the accessory (1302) is pre-equipped with information about at least one pill (1303).

7. The system of claim 5, wherein the accessory is programmed to monitor and/or coordinate delivery of substances and/or services by a plurality of pills and is programmed with respective encryption related information about at least two pills and each message (1401) is encrypted using respective encryption information associated with a particular pill.

8. The system of claim 5, wherein the device is programmed with respective encryption related information about at least two pills and each message (1401) is encrypted using respective encryption information associated with a particular pill.

9. The system according to claim 5, wherein secure communication is established between said accessory and at least one pill from security related information about the wearable accessory stored in the pill (818) and/or from security related information about the pill stored in the wearable accessory (606, 814).

10. The system according to claim 5, wherein establishing secure communication comprises:
associating one wearable accessory (808) with a set of pills (809); and
programming the wearable accessory (808) with security information regarding the pills (809).

11. The system according to claim 5, wherein data regarding medical substances or services to be delivered by the pills is stored in the wearable accessory.

12. The system according to claim 5, wherein the establishing communication comprises adding information about at least one new pill to a wearable accessory previously programmed with information about at least one prior pill.

13. A method comprising executing the following operations in a medical security system:
maintaining a close proximity between at least one wearable accessory (103) and a human body (100);
passing at least one pill (101) through the alimentary tract of the body (100);
performing a security related operation (703), to verify identity of either the wearable accessory to the pill, or to verify identity of the pill to the wearable accessory, or to verify the identity of the pill and the wearable accessory to each other.

14. A medium readable by a data processing device and embodying executable code for causing the device to perform operations, the operations including:
receiving information (821, 813) regarding at least one pill (809) and at least one wearable accessory (808);
establishing communication with the pill and/or the wearable accessory; and
programming either the wearable accessory or the pill or both with information (818, 814) about the other.

15. An apparatus (103) comprising:
at least one wireless transceiver;
at least one storage device embodying data and/or executable code in a machine readable form;
at least one processor adapted to perform operations, the operations comprising:
maintaining identifying information (1201) about at least one pill;
sending and/or receiving information (1202) relating to delivery of at least one service and/or substance to and/or from the pill, while the pill is in a human body;
determining (1204) that further communication with the pill is no longer useful; and
deleting (1205) the identifying information about the at least one pill that is no longer useful.
